(19) United States
(12) Patent Application Publication
(10) Pub. No.: US 2020/0337835 A1
(43) Pub. Date: Oct. 29, 2020

(54) MEDICAL IMPLANT

(71) Applicant: Medical and pharmaceutical Industry Technology and Development Center, New Taipei City (TW)

(72) Inventors: Sheng-Chung Cheng, New Taipei City (TW); Han-Tang Liu, New Taipei City (TW); Chung-Chih Cheng, Taipei City (TW); Jou-Wen Chen, New Taipei City (TW); Yong-Guei Chen, Taipei City (TW); Chih-Chiang Yang, Taipei City (TW); Wei-Ting Huang, Hsinchu City (TW); Yao-Chung Yu, Taipei City (TW); Ting-Shu Lin, New Taipei City (TW)

(21) Appl. No.: 16/861,222

(22) Filed: Apr. 29, 2020

Related U.S. Application Data
(63) Continuation of application No. PCT/CN2020/083821, filed on Apr. 8, 2020.
(60) Provisional application No. 62/839,793, filed on Apr. 29, 2019.

(51) Int. Cl. A61F 2/18; A61F 5/08

(52) U.S. Cl. CPC A61F 2/186; A61F 5/08; A61F 2210/0004; A61F 2220/0025; A61F 2230/0017

(57) ABSTRACT

A medical implant has a center axis and includes first and second flexible waved strands disposed around the center axis. The second flexible waved strand is in spatial communication with the first flexible waved strand to form a plurality of first unit shapes and a plurality of second unit shapes. Therein, the first unit shapes and the second unit shapes are staggered around the center axis. The first unit shapes are coupled to the second unit shapes to cause the first and second flexible waved strands to move substantially along the center axis. The first and second flexible waved strands together define a self-anchoring configuration in a radial direction perpendicular to the center axis so that a ratio of a von Mises stress to an axial displacement of the medical implant during an implant compression of the medical implant is greater than 0.1 and less than 30.

### CROSS REFERENCE TO RELATED APPLICATIONS

This application is a continuation of PCT Application No. PCT/CN2020/083821 filed on 2020 Apr. 8, which claims the benefit of U.S. Provisional Application No. 62/839,793 filed on 2019 Apr. 29, the entire contents of which are incorporated herein by reference.

### BACKGROUND OF THE INVENTION

### 1. Field of the Invention

The present invention relates to a medical implant, and more particularly, to a biodegradable, low adherent medical implant that is placed within a lumen of a patient.

### 2. Description of the Prior Art

Chronic rhinosinusitis (CRS) is a common condition characteristic of mucosal inflammation within nasal passages for at least 12 weeks. CRS is divided into two clinical categories: rhinosinusitis with nasal polyps (CRSwNP) and chronic rhinosinusitis without nasal polyps (CRSwNP).

Patients with CRS may require medical management to alleviate disease aggravation and minimize the risk of associated disease variants. For patients with CRSwNP, functional endoscopic sinus surgery (FESS) is an increasingly popular medical management solution. Although FESS has undergone refinement over time, the most common surgical complication remains persistent inflammation and disease recurrence. As such, careful post-operative care management to address appropriate inflammation recurrence is desired.

Mucous membrane is prone to inflammation and adhesion during the recovery of the nasal cavity, leading to the proliferation of scar tissue, which in turn stimulates the growth of nasal polyps and causes the recurrence of chronic sinusitis. Currently available post-operative care management prefers using pharmaceutical agents, including as an example steroids, to address local inflammation occurrence, as well as sinus stickiness.

There is therefore a need for a low adherent, implantable implant that has sufficient strength and other mechanical and drug release properties that are necessary to effectively treat the medical conditions for which they are used.

### SUMMARY OF THE INVENTION

An objective of the invention is to provide a medical implant, which has sufficient strength and uses a plurality of flexible waved strands to define a self-anchoring configuration suitable for implantation.

A medical implant according to the invention has a center axis and includes a first flexible waved strand and a second flexible waved strand which are disposed around the center axis. The second flexible waved strand is in spatial communication with the first flexible waved strand to form a plurality of first unit shapes and a plurality of second unit shapes. Therein, the first unit shapes and the second unit shapes are staggered around the center axis. The first unit shapes are coupled to the second unit shapes to cause the first and second flexible waved strands to move substantially along the center axis. The first and second flexible waved strands together define a self-anchoring configuration in a radial direction perpendicular to the center axis so that a ratio of a von Mises stress to an axial displacement of the medical implant during an implant compression of the medical implant is greater than 0.1 and less than 30. Therein, the von Mises stress is expressed in megapascals (MPa), and the axial displacement is expressed in millimeter (mm). Thereby, the medical implant according to the invention is so flexible as to be smoothly delivered through a cannula (e.g. of a delivery device) and then, due to its resilience, can expand with maintaining sufficient strength so as to maintain patency in the lumen after implanted.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a medical implant according to an embodiment.

FIG. 2 is a schematic diagram illustrating a portion of the medical implant in FIG. 1.

FIG. 3 is a schematic diagram illustrating the medical implant in FIG. 1 when compressed radially.

FIG. 4 is a schematic diagram illustrating a portion of a medical implant according to another embodiment.

FIG. 5 is a schematic diagram illustrating a portion of a medical implant according to another embodiment.

FIG. 6 is a schematic diagram illustrating a strand according to an embodiment.

FIG. 7 is a schematic diagram illustrating the strand in FIG. 6 coated with a topcoat.

FIG. 8 is a schematic diagram illustrating an arrangement of the filaments of the strand in FIG. 7 according to an embodiment.

FIG. 9 is a schematic diagram illustrating the strand in FIG. 8 coated with a topcoat.

FIG. 10 is a schematic diagram illustrating a strand with a hexagon section according to an embodiment.

FIG. 11 is a schematic diagram illustrating a medical implant according to another embodiment.

FIG. 12 is a schematic diagram illustrating the medical implant in FIG. 11 when compressed radially.

FIG. 13 is a schematic diagram illustrating a medical implant according to another embodiment.

FIG. 14 is a schematic diagram illustrating a medical implant according to another embodiment.

FIG. 15 is a schematic diagram of maximum von Mises stresses and maximum principal stress with respect to internal angle ratios.

FIG. 16 is a schematic diagram illustrating the trendline for influence of internal angle ratios on maximum von Mises stress.

FIG. 17 is a schematic diagram illustrating the trendline for influence of internal angle ratios on maximum principal stress.

FIG. 18 is a schematic diagram of maximum von Mises stresses and maximum principal stress with respect to radius of curvature ratios.

FIG. 19 is a schematic diagram illustrating the trendline for influence of radius of curvature ratios on maximum von Mises stress.

FIG. 20 is a schematic diagram illustrating the trendline for influence of radius of curvature ratios on maximum principal stress.

### DETAILED DESCRIPTION

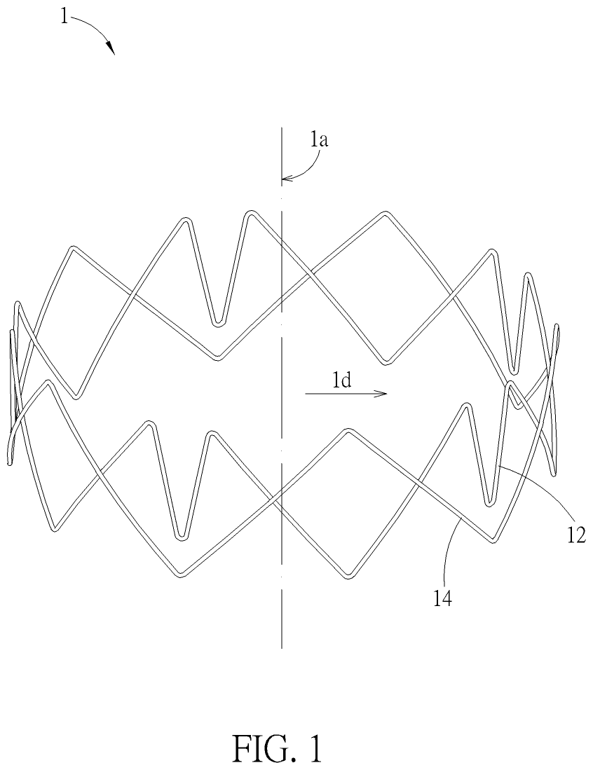

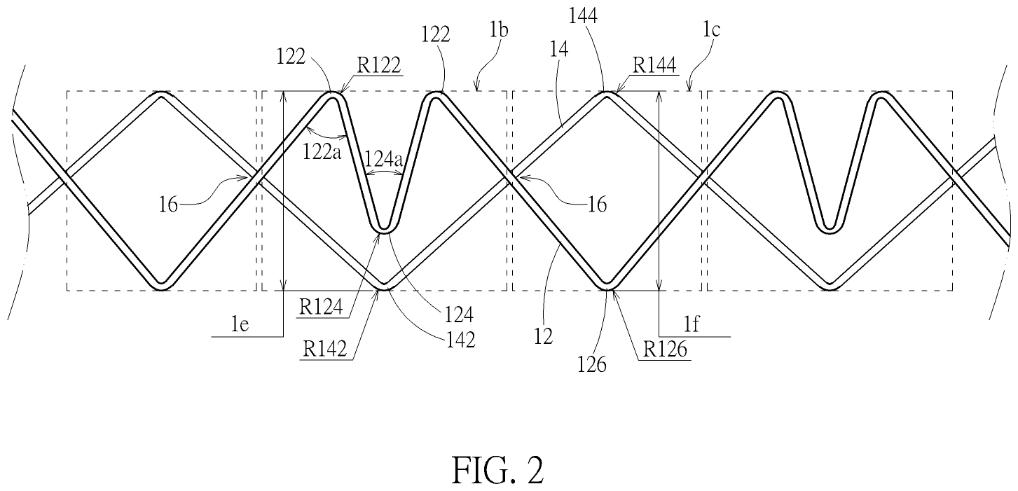

Please refer to FIG. 1 to FIG. 3. A medical implant 1 according to an embodiment has a center axis 1a (indicated by a chain cline in FIG. 1) and includes a first flexible waved strand 12 and a second flexible waved strand 14 which are disposed around the center axis 1a. The first and second flexible waved strands 12 and 14 are in spatial communication with each other to form a plurality of first unit shapes 1b (indicated by dashed frames in FIG. 2) and a plurality of second unit shapes 1c (indicated by dashed frames in FIG. 2). The first unit shapes 1b and the second unit shapes 1c are staggered around the center axis 1a. The first unit shapes 1b are coupled to the second unit shapes 1c so that the first and second flexible waved strands 12 and 14 can be moved substantially along the center axis 1a. The medical implant 1 is flexible to be radially compressible (and extensible along the center axis 1a) and is also radially self-expandable (and contractible along the center axis 1a). Thereby, the medical implant 1 has a compressible configuration and a self-anchoring configuration which are structured and established by the first and second flexible waved strands 12 and 14.

In the embodiment, the medical implant 1 as a whole is flexible in structure to a certain extent. By choosing appropriate material as the first and second flexible waved strands 12 and 14, the elasticity of the medical implant 1 can be further increased. In the medical implant 1, the first and second flexible waved strands 12 and 14 are elastic, so that the medical implant 1 can elastically extends along the center axis 1a and shrink in a direction opposite to a radial direction 1d (indicated exemplarily by an arrow in FIG. 1; more precisely, the radial direction 1d refers to directions pointing from the center axis 1a toward all sides) perpendicular to the center axis 1a when the medical implant 1 is compressed in the direction opposite to a radial direction 1d, and the medical implant 1 can elastically shrink along the center axis 1a and expand in the radial direction 1d after the constraint on the compressed medical implant 1 is removed.

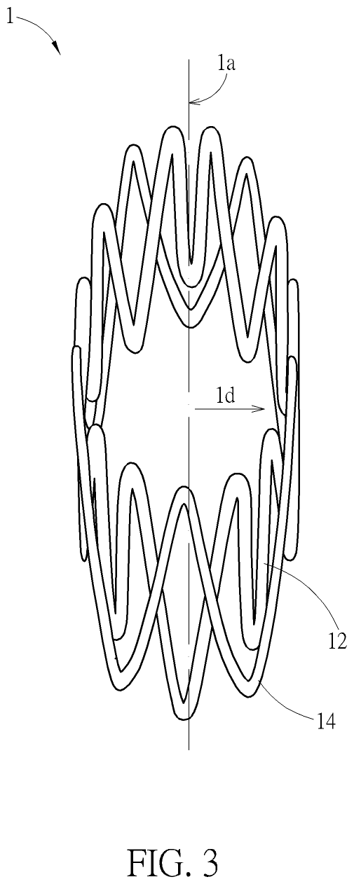

When in use, the medical implant 1 at an un-compressed status (as shown by FIG. 1) can be radially compressed to extend along the center axis 1a and reduce the overall size perpendicular to the center axis 1a to be at a compressed status (as shown by FIG. 3), which is conducive to delivery through a cannula (e.g. of a delivery device). Then, after located to a lumen through the delivery device, due to its resilience (which produces an outward force resulting in a tendency to return to the un-compressed status), the medical implant 1 can expand with maintaining sufficient strength and abut against the inner wall surface of the lumen so as to maintain patency in the lumen (i.e. self-expanding) after implanted. In practice, for example, the lumen can be the interior of a blood vessel (e.g. arteries or vascular cavities), the interior of the gastrointestinal tract (e.g. esophagus, intestine), the passage of the respiratory system (e.g. bronchi, paranasal sinuses), the passage of the auditory system (e.g. ear canals), the interior of the urinary collecting duct system (e.g. prostate gland, urethra, biliary tract), and so on.

The compressible configuration and the self-anchoring configuration work in such a way that the compressible configuration of the medical implant 1 is the structural portion that is adaptive to the internal volumetric variation of the lumen, while the self-anchoring configuration is the structural portion that remains unchanged once attached to the inner wall surface of the lumen. In the embodiment, the first and second flexible waved strands 12 and 14 together define the self-anchoring configuration in the radial direction 1d so that a ratio of a von Mises stress to an axial displacement (i.e. displacement along the center axis 1a) of the medical implant 1 during an implant compression of the medical implant is greater than 0.1 and less than 30. Therein, the von Mises stress is expressed in megapascals, and the axial displacement is expressed in millimeter. Thereby, the medical implant 1 can better fit to the inner wall surface of the lumen without substantially damaging the inner wall surface, while still maintaining a certain structural strength. In other words, the medical implant 1 is operative to distribute pressure evenly on the inner wall surface.

In the embodiment, the medical implant 1 is provided in form of a crown structure and shows a substantially tubular configuration. The first unit shape 1b and the second unit shape 1c are mutually exclusive in shape, which is suitable for adjusting and designing the stress distribution of the medical implant 1. The first flexible waved strand 12 and the second flexible waved strand 14 overlap and are connected through a plurality of joints 16. The joints 16 can be achieved by glue or other methods capable of connecting the adjacent strands together. The joints 16 are located between the first unit shapes 1b and the second unit shapes 1c. In the embodiment, there is one joint 16 between any two adjacent first and second unit shapes 1b and 1c; however, it is not limited thereto in practice. For example, it is practicable to join the first and second flexible waved strands 12 and 14 by every two or more unit shapes (including at least one first unit shape 1b and at least one second unit shape 1c) or other the same or different intervals.

In the embodiment, in the view of FIG. 2, the first unit shape 1b includes two peaks 122 and one trough 124 of the first flexible waved strand 12, and a trough 142 of the second flexible waved strand 14. The first unit shape 1b is heart-shaped. Furthermore, the trough 142 is aligned with the trough 124 in a direction parallel to the center axis 1a; however, it is not limited thereto in practice. The second unit shape 1c includes one trough 126 of the first flexible waved strand 12 and one peak 144 of the second flexible waved strand 14. The second unit shape 1c is diamond. Furthermore, the trough 126 is aligned with the peak 144 in a direction parallel to the center axis 1a; however, it is not limited thereto in practice.

Furthermore, in the embodiment, in the first unit shape 1b, the trough 124 has an internal angle 124a that can be designed to be less than 87 degrees and not less than 3 degrees; the peak 122 has an internal angle 122a that can be designed to be less than 87 degrees and not less than 4 degrees. However, it is not limited thereto in practice. Furthermore, when a ratio of the internal angle 124a of the trough 124 to the internal angle 122a of the peak 122 is about 0.5, the von Mises stress reaches a relatively lower value; for example, the von Mises stress is about 160 MPa as the axial displacement is about 13 mm, and the Young's Modulus of the material for the first and second flexible waved strands 12 and 14 is about 25 GPa.

Furthermore, in the embodiment, a radius of curvature (i.e. labeled as R122, R124, R126) of an outer edge of any curvilinear arc (i.e. any of the peaks 122 and the troughs 124 and 126) of the first flexible waved strand 12 is less than or equal to a radius of curvature (i.e. labeled as R142, R144) of an outer edge of any curvilinear arc (i.e. any of the trough 142 and the peak 144) of the second flexible waved strand 14. In practice, the radius of curvature R122, R124 and R126 can be designed to be less than 15 mm and not less than 0.35 mm. The radius of curvature R142 and R144 can be designed to be less than 15 mm and not less than 0.35 mm. However, it is not limited thereto in practice. Furthermore, when a ratio of the radius of curvature R124 of the trough 124 to the radius of curvature R122 of the peak 122 is about 1, the von Mises stress reaches a relatively lower value; for example, the von Mises stress is about 160 MPa as the axial displacement is about 13 mm, and the Young's Modulus of the material for the first and second flexible waved strands 12 and 14 is about 25 GPa.

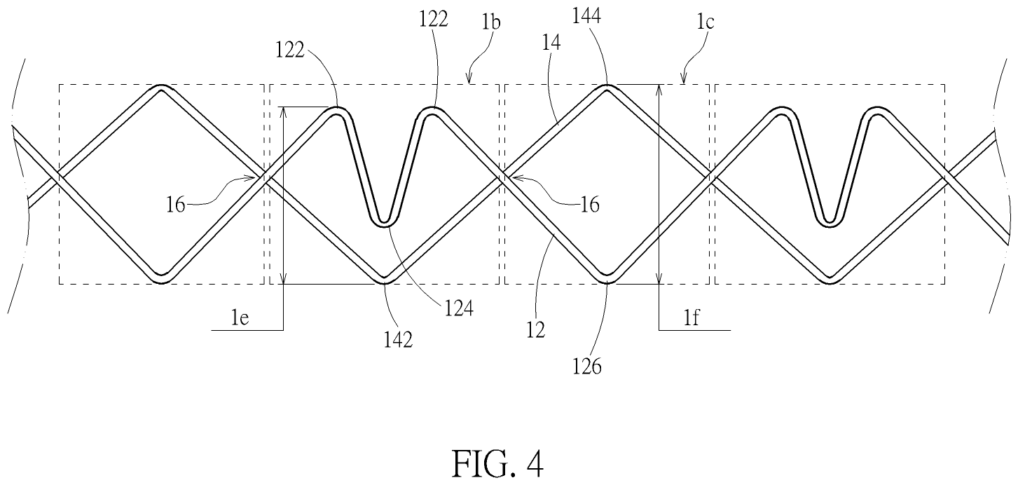

Furthermore, in the embodiment, the first unit shape 1b has a first length 1e along the center axis 1a. The second unit shape 1c has a second length 1f along the center axis 1a. The first length 1e is substantially equal to the second length 1f. However, it is not limited thereto in practice. In practice, it is practicable for the first unit shape 1b and the second unit shape 1c to have different lengths along the center axis 1a; that is, the first length 1e is different to the second length 1f. For example, as shown by FIG. 4, the first length 1e is less than the second length 1f.

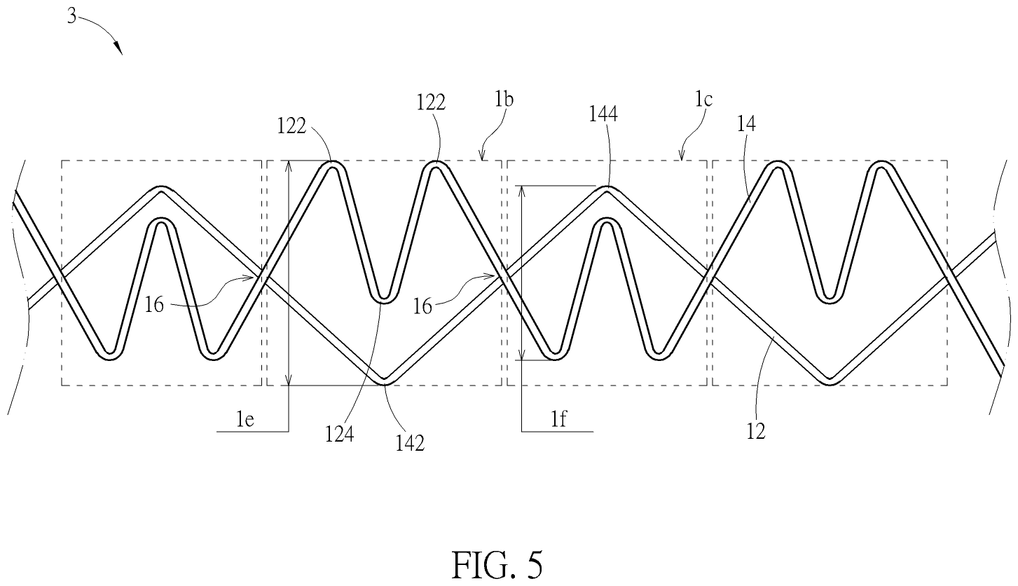

In the medical implant 1, the first and second unit shapes 1b and 1c are mutually exclusive in shape and are heart-shaped and diamond respectively, but it is not limited thereto in practice. For example, please refer to FIG. 5, which shows a side view of a portion of a medical implant 3 according to another embodiment. The medical implant 3 is structurally similar to the medical implant 1, so the medical implant 3 uses the reference numbers of the medical implant 1 for description simplification. For other descriptions about the medical implant 3, please refer to the relevant descriptions of the medical implant 1 and variation thereof, which will not be described in addition. In the medical implant 3, the first unit shape 1b is heart-shaped, and the second unit shape 1c is reverse heart-shaped. In logic, the first and second unit shapes 1b and 1c are still mutually exclusive in shape. Furthermore, the first length 1e is greater than the second length 1f. However, it is not limited thereto in practice. Similarly, the first unit shape 1b and the second unit shape 1c can be designed to have the same length or different lengths along the center axis 1a in practice.

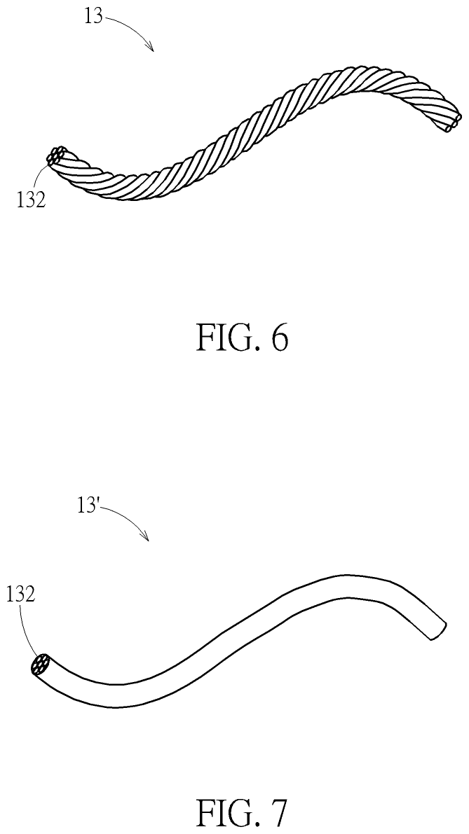

In practice, in the medical implants 1 and 3, one or both of the first and second flexible waved strands 12 and 14 can be made of biodegradable polymer, ceramic, metal alloy or a combination thereof. One or both of the first and second flexible waved strands 12 and 14 can be constructed of a strand 13 that includes a plurality of filaments 132, as shown by FIG. 6. The filament 132 can be monofiber or multifiber. The monofiber or multifiber can be biodegradable. The filament 132 can be made of a polymeric material, or a polymer matrix reinforced with fibers. The first and second flexible waved strands 12 and 14 do not need to be made of the same material. When the first and second flexible waved strands 12 and 14 are biodegradable, the first and second flexible waved strands 12 and 14 are preferably fully absorbed within about one year of placement within a patient, more preferably within about six months of placement within a patient, and most preferably within about one month of placement within a patient.

Examples of biodegradable polymers that are useful in the present invention include poly lactic acid (PLA), poly glycolic acid (PGA), poly trimethyllene carbonate (PTMC), poly caprolactone (PCL), poly dioxanone (PDO), poly (lactic-co-glycolic acid) (PLGA), chitosan, hydroxypropylmethylcellulose (HPMC), hydroxypropyl cellulose (HPC), gelatin, poly (vinyl alcohol) (PVA), polyvinylpyrrolidone (PVP), polyethylene glycol (PEG), polyethersulfone (PES), and copolymers thereof.

Examples of metal alloy that are useful in the present invention include magnesium alloy, iron alloy, memory alloy metal.

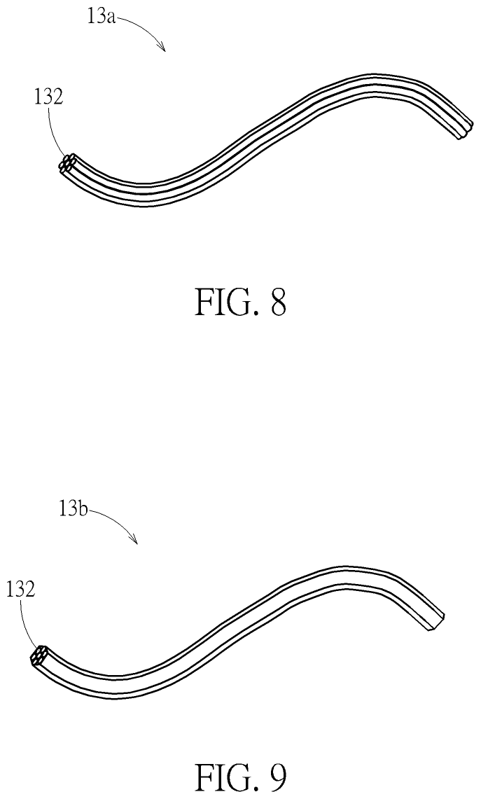

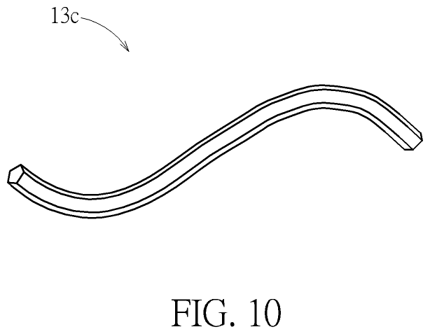

As shown by FIG. 6, the strand 13 includes seven filaments 132 that are twisted into a bundle with a predetermined section. The strand 13 as a whole shows a substantially circular section; for any single section, it shows a hexagon. In practice, the strand 13 can be coated with a topcoat as the strand 13′ as shown by FIG. 7. In another embodiment as shown by FIG. 8, a strand 13a also includes seven filaments 132 bundled without twisting. The strand 13a has a hexagon section. In practice, the strand 13a also can be coated with a topcoat as the strand 13b as shown by FIG. 9. In another embodiment as shown by FIG. 10, a strand 13c includes a plurality of filaments (i.e. the above filaments) bundled to have a pentagon section; therein, the strand 13c is shown by a single part for drawing simplification. In addition, for the above strand 13, 13′, 13a, 13b and 13c, the filament 132 can be hollow, solid, or porous. Furthermore, the strand 13 also can be a monofilament in practice. The medical implants 1 and 3 can be provided by weaving filaments (provided by extrusion), injection molding, 3D printing, and so on.

In addition, by designing the above structural dimensions and material of the medical implants 1 and 3, the stress distribution of the medical implants 1 and 3 is controllable or adjustable for low adhesion, for example, so that polyp growth can be inhibited and sinusitis recurrence can be minimized. The medical implant 1, for example, can deliver one or more therapeutic agents at the site of implantation. Therapeutic agent may be applied to one or more strands 12 and 14 for delivery therefrom in a number of ways. In one example, therapeutic agent is embedded within a coating that adheres to one or more individual strands 12 and 14 of the medical implant 1, preferably conformal to the contours of the strands 12 and 14. In some embodiments, the coating may be fully conformal to the contours of the strands 12 and 14. In some embodiments, the coating may be partially conformal to the contours of the strands 12 and 14. In yet some other embodiments, the coating may be non-conformal to the contours of the strands 12 and 14. The coating is preferably made from a biodegradable polymer. The biodegradable polymer may be admixed with therapeutic agent such that the agent is eluted from the polymer over time, or is released from the coating as it degrades in vivo. The formation of the coating can be achieved by partially or fully spraying or immersing, or other methods.

In practice, therapeutic agent can be any agent that can deliver desired therapeutic effects for appropriate medical treatment scheme. Therapeutic agent is selected alone or in combination from steroids (such as mometasone furoate, fluticasone, fluticasone propionate, beclometasone), antihistamines (such as azelastine), analgesic agents, antibiotic agents, and anti-inflammatory agents (such as budesonide, triamcinolone).

Coating or areas containing one or more therapeutic agents can be applied to the medical implant 1 by any appropriate method, including but not limited to spraying, electrospraying, dipping, flowing and chemical vapor deposition. The coating or areas containing one or more therapeutic agents can be a single layer or multiple layers. The layering established by the coating or areas containing one or more therapeutic agents can be composed of a first coating, a second coating, or a combination thereof. The terms “first” and “second” are used to distinguish them from each other, and do not necessarily represent sequence during coating process. Examples of the components in the layer (s) that are useful for the medical implant 1 include a diluent, a binder, a disintegrant, a lubricant, a glidant, or one or more therapeutic agent. In addition, therapeutic agents and the medical implant 1 can be combined by any appropriate method, including but not limited to mixing, coating, blending, and diffusion. Alternatively, one or more therapeutic agents can be embedded or compounded into the implant.

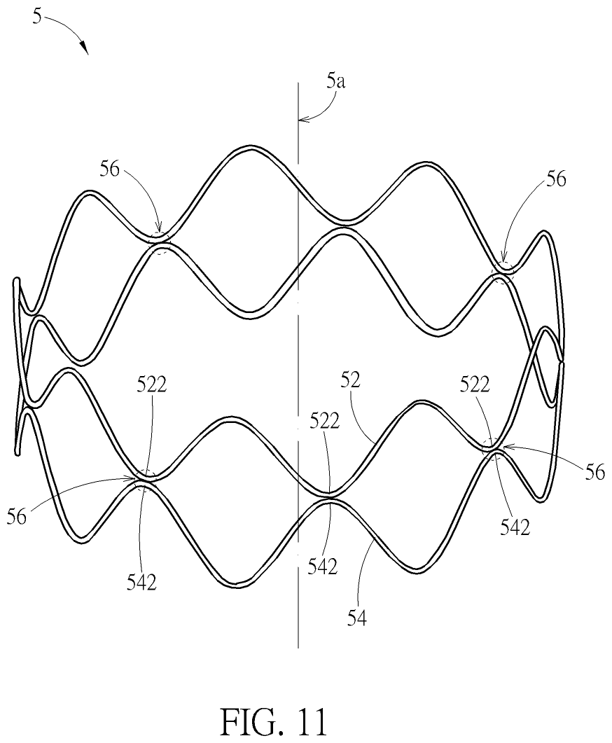

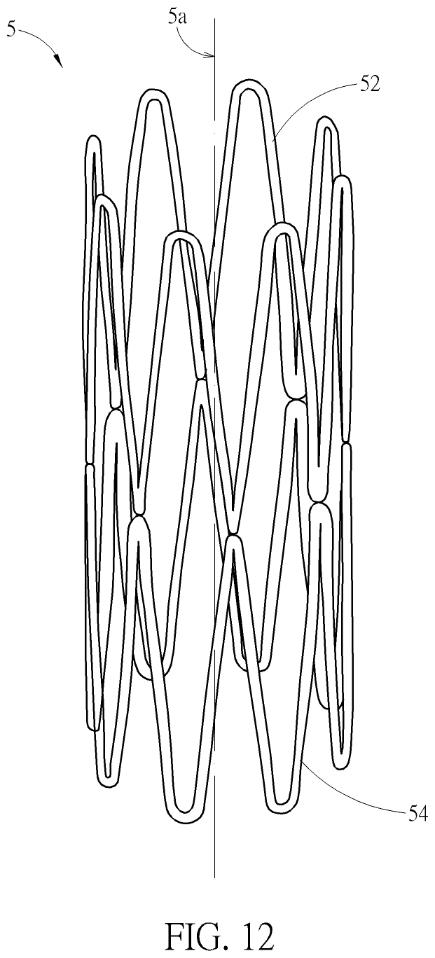

In addition, in the medical implants 1 and 3, the first and second flexible waved strands 12 and 14 form two kinds of unit shapes 1b and 1c; however, it is not limited thereto in practice. For example, the first and second flexible waved strands can form more kinds of unit shapes, which facilitates designing the stress distribution of the medical implant. Furthermore, in the medical implants 1 and 3, the first flexible waved strand 12 and the second flexible waved strand 14 overlap; however, it is not limited thereto in practice. Please refer to FIG. 11 and FIG. 12. A medical implant 5 according to an embodiment has a center axis 5a (indicated by a chain cline in FIG. 11 and FIG. 12) and includes a first flexible waved strand 52 and a second flexible waved strand 54 which are disposed around the center axis 5a and staggered along the center axis 5a. The first flexible waved strand 52 and the second flexible waved strand 54 adjoin each other along the center axis 5a without overlapping. Similar to the medical implants 1 and 3, the medical implant 5 is flexible to be radially compressible (and extensible along the center axis 5a) and is also radially self-expandable (and contractible along the center axis 5a). Thereby, the medical implant 5 has a compressible configuration and a self-anchoring configuration which are structured and established by the first and second flexible waved strands 52 and 54. Therein, the medical implant 5 can be compressed as shown by FIG. 12. Similarly, the medical implant 5 can deliver one or more therapeutic agents at the site of implantation. For other descriptions about the medical implant 5, please refer to the relevant descriptions of the medical implant 1 and variation thereof, which will not be described in addition.

In the embodiment, in the view of FIG. 11, the first and second flexible waved strands 52 and 54 are connected through a plurality of joints 56 (of which the location is indicated by dashed circles in FIG. 11) at every two troughs 522 of the first flexible waved strand 52 and corresponding peaks 542 of the second flexible waved strand 54. The joints 56 can be achieved by glue, interweaving or other methods capable of connecting the adjacent strands together. Alternatively, the first and second flexible waved strands 52 and 54 also can be connected at each troughs 522 of the first flexible waved strand 52 and corresponding peaks 542 of the second flexible waved strand 54, or at every more than two troughs 522 of the first flexible waved strand 52 and corresponding peaks 542 of the second flexible waved strand 54. The material and production of the first and second flexible waved strands 52 and 54 can refer to that of the medical implant 1 and will not be described in addition.

Furthermore, in practice, it is practicable to add more waved strands to the medical implant 5 to be a medical implant 6 with a longer axial length along its center axis 6a, as shown by FIG. 13; therein, the medical implant 6 includes one waved strand 62 more than the medical implant 5. The connection of the waved strand 62 with the adjacent waved strand 54 can be achieved by the same way as that of the first and second flexible waved strands 52 and 54.

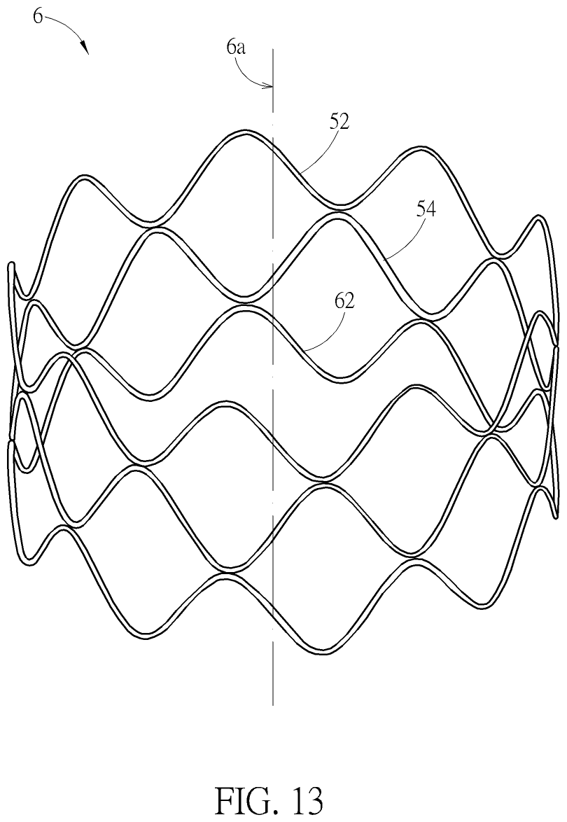

Similarly, it is practicable to add more waved strands to the medical implant 1 to be a medical implant 7 with a longer axial length along its center axis 7a, as shown by FIG. 14; therein, the medical implant 7 is equivalent to a combination of two medical implants 1, of which the connection can be achieved by the same way as that of the first and second flexible waved strands 52 and 54. In addition, it is practicable to connect the medical implants 1, 3 and 5 in series (i.e. along the center axis) with different numbers.

### Example

In the following, examples designed in accordance with the medical implant described above are tested in comparison with a Comparative Example. A variety of internal angle ratios and radius of curvature ratios as examples of the medical implant were measured to determine their influence on the medical implant's yielding conditions, indexed herein by maximum von Mises stress and maximum principal stress. Materials with Young's modulus at 200 MPa, 25 GPa, and 50 GPa were used for examples of the medical implant.

Table 1 below shows respective sizes of the samples in Example (e.g. the medical implant 1 described above) and Comparative Example (e.g. a device 1722 shown by FIG. 17C of U.S. Ser. No. 10/010,651), including the von Mises stress at the time of compression with a radial displacement by an axial displacement of 25% of the diameter of the implant, and applied load.

TABLE 1
Compression resistance to perpendicularly
applied load

| | | Comparative |
|---|---|---|
| | Example | Example |
| Displacement (mm) | 13 | 13 |
| Von Mises Stress (MPa) | 182 | 195 |
| Force (N) | 0.14047 | 0.14159 |

Influence of Internal Angle Ratio or Radius of Curvature Ratio on Maximum Von Mises Stress and Maximum Principal Stress

Mechanical structure computer analysis was performed on the medical implant to determine the maximum von Mises stress and maximum principal stress which occur during simulated compression of the component. This analysis may be supplemented with empirical testing.

It was confirmed that internal angle ratio (e.g. the ratio of the internal angle 124a of the trough 124 to the internal angle 122a of the peak 122, shown by FIG. 2) of 1:2 was the more preferred for medical implants having Young's modulus of 50 GPa, 25 GPa, and 200 MPa. Radius of curvature (e.g. the ratio of the radius of curvature R124 to the radius of curvature R122, shown by FIG. 2) of 1:1 was the more preferred for medical implants having Young's modulus of 50 GPa, 25 GPa, and 200 MPa.

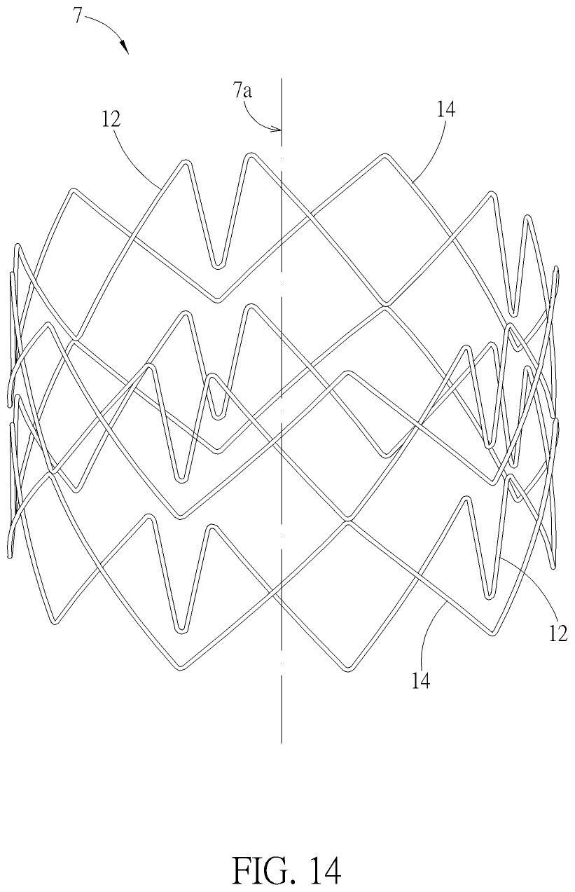

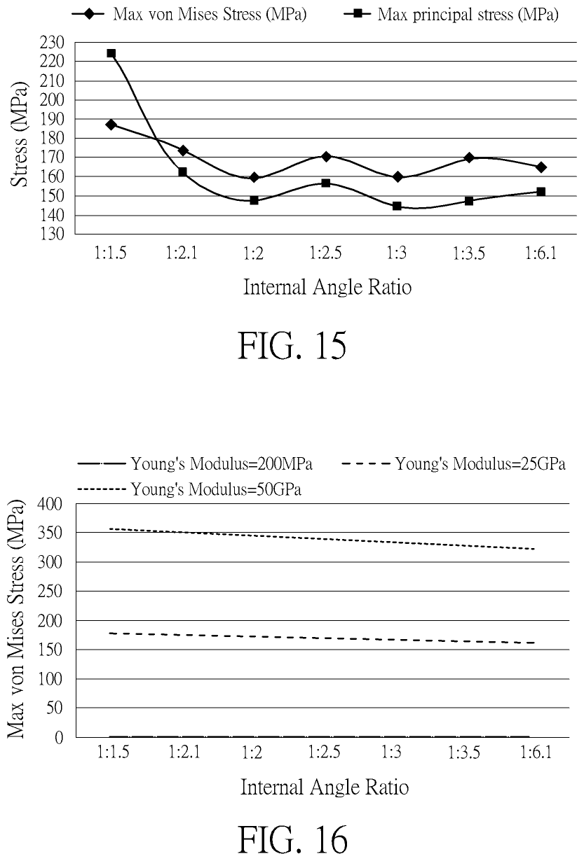

Results from the simulated compression demonstrated that the maximum von Mises stresses with respect to internal angle ratios of 1:1.5, 1:2.1, 1:2, 1:2.5, 1:3, 1:3.5, and 1:6.1, as shown by FIG. 15. Additionally, 1:2 is the most preferred internal angle ratio in terms of manufacturability.

FIG. 16 shows the trendline for influence of internal angle ratios on maximum von Mises stress. The compression test was performed using finite element analysis on internal angle ratios of 1:1.5, 1:2.1, 1:2, 1:2.5, 1:3, 1:3.5, and 1:6.1 delineated for medical implants manufactured from materials having Young's Modulus of 200 MPa, 25 GPa, and 50 GPa. Results show that the maximum von Mises stress generally decreases as the internal angle ratio increases, and the maximum von Mises stress level decreases with decreasing Young's modulus level. It is also shown that the magnitude of reduction in maximum von Mises stress with increasing internal angle ratio is more noticeable with Young's modulus of 50 GPa than 25 GPa, and 200 MPa respectively.

FIG. 17 shows the trendline for influence of internal angle ratios on maximum principal stress. The compression test using finite element analysis was performed on internal angle ratios of 1:1.5, 1:2.1, 1:2, 1:2.5, 1:3, 1:3.5, and 1:6.1 delineated for medical implants manufactured from materials having Young's Modulus of 200 MPa, 25 GPa, and 50 GPa. Results show that the maximum principal stress generally decrease as the internal angle ratio increases, and the maximum principal stress level decreases with decreasing Young's modulus level. It is shown that the reduction in maximum principal stress with increasing internal angle ratio is more noticeable with Young's modulus of 50 GPa than 25 GPa, and 200 MPa respectively.

The data in FIGS. 16 and 17 are also shown in the following tables 2 to 4.

TABLE 2
Young's Modulus: 25 GPa

| | | | | | | |
|---|---|---|---|---|---|---|
| Internal | 1:1.5 | 1:2.1 | 1:2 | 1:2.5 | 1:3 | 1:3.5 |
| Angle Ratio | | | | | | |
| Actual Values | 37:55.5 | 24:51 | 25:50 | 18.5:50 | 15:48 | 12:47 |
| Max von Mises | 187.45 | 174.07 | 159.92 | 170.59 | 160.24 | 169.91 |
| Stress (MPa) | | | | | | |
| Max principal | 223.92 | 162.48 | 147.65 | 156.61 | 144.59 | 147.42 |
| stress (MPa) | | | | | | |

TABLE 3
Young's Modulus: 200 MPa

| | | | | | | |
|---|---|---|---|---|---|---|
| Internal | 1:1.5 | 1:2.1 | 1:2 | 1:2.5 | 1:3 | 1:3.5 |
| Angle Ratio | | | | | | |
| Actual Values | 37:55.5 | 24:51 | 25:50 | 18.5:50 | 15:48 | 12:47 |
| Max von Mises | 1.5104 | 1.3926 | 1.2794 | 1.3647 | 1.2819 | 1.3593 |
| Stress (MPa) | | | | | | |
| Max principal | 1.7914 | 1.2998 | 1.1812 | 1.2529 | 1.1567 | 1.1793 |
| stress (MPa) | | | | | | |

TABLE 4
Young's Modulus: 50 GPa

| | | | | | | |
|---|---|---|---|---|---|---|
| Internal | 1:1.5 | 1:2.1 | 1:2 | 1:2.5 | 1:3 | 1:3.5 |
| Angle Ratio | | | | | | |
| Actual Values | 37:55.5 | 24:51 | 25:50 | 18.5:50 | 15:48 | 12:47 |
| Max von Mises | 377.60 | 348.14 | 319.84 | 341.17 | 320.48 | 339.81 |
| Stress (MPa) | | | | | | |
| Max principal | 447.85 | 324.96 | 295.29 | 313.22 | 289.18 | 294.84 |
| stress (MPa) | | | | | | |

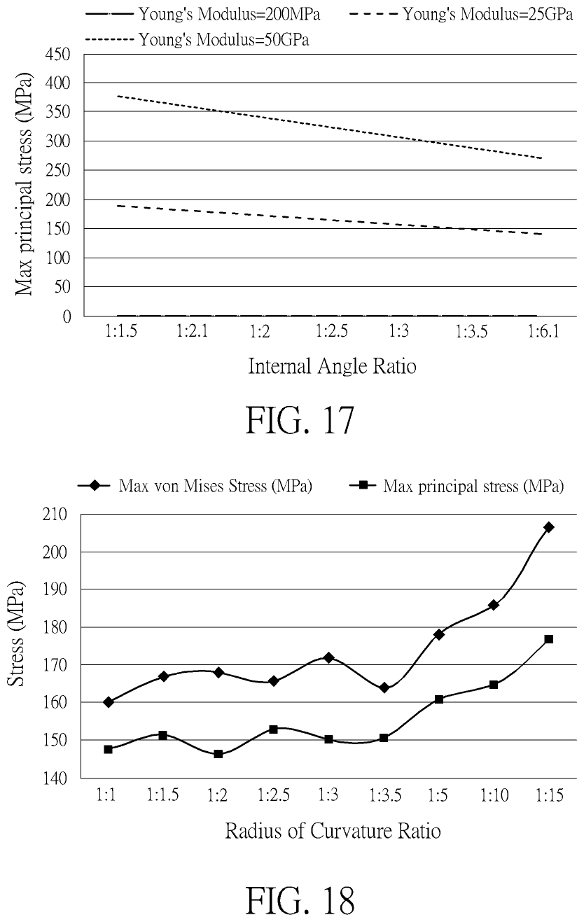

FIG. 18 shows the results from the compression test using finite element analysis for the maximum von Mises stresses as well as maximum principal stresses with respect to radius of curvature ratios of 1:1, 1:1.5, 1:2, 1:2.5, 1:3, 1:3.5, 1:5, 1:10, and 1:15.

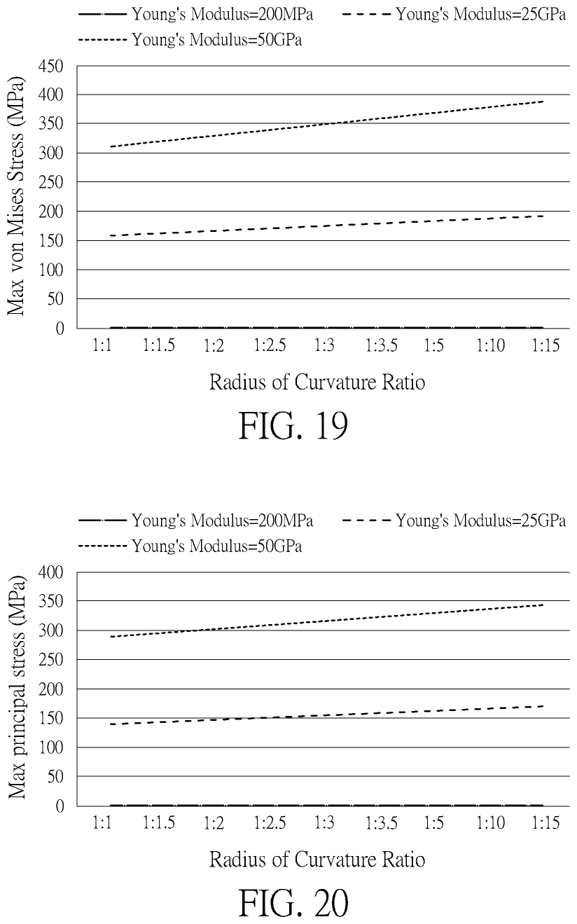

The trendline for influence of internal angle ratio on maximum von Mises stress in a compression test using finite element analysis shows that the maximum von Mises stress generally increases as the radius of curvature ratio increases, as shown by FIG. 19. The maximum von Mises stress level decreases with increasing respective Young's modulus level. It is also shown that the magnitude of increase in maximum von Mises stress with increasing radius of curvature ratio is more noticeable with Young's modulus of 50 GPa than 25 GPa and 200 MPa.

The trendline for influence of radius of curvature ratio on maximum principal stress in a compression test using finite element analysis shows that the maximum principal stress generally increases as the radius of curvature ratio increases, as shown by FIG. 20. The maximum principal stress level decreases with increasing respective Young's modulus level. It is also shown that the magnitude of increase in maximum principal stress with increasing radius of curvature ratio is more noticeable with Young's modulus of 50 GPa than 25 GPa and 200 MPa.

The data in FIGS. 19 and 20 are also shown in the following tables 5 to 7.

TABLE 5
Young's Modulus: 25 GPa

| | | | | | | |
|---|---|---|---|---|---|---|
| Radius of | 1:1.0 | 1:1.5 | 1:2.0 | 1:2.5 | 1:3.0 | 1:3.5 |
| Curvature Ratio | | | | | | |
| Max von Mises | 159.92 | 166.78 | 167.95 | 165.63 | 171.83 | 163.85 |
| Stress (MPa) | | | | | | |
| Max principal | 147.65 | 151.38 | 146.51 | 152.89 | 150.26 | 150.74 |
| stress (MPa) | | | | | | |

TABLE 6
Young's Modulus: 200 MPa

| | | | | | | |
|---|---|---|---|---|---|---|
| Radius of | 1:1.0 | 1:1.5 | 1:2.0 | 1:2.5 | 1:3.0 | 1:3.5 |
| Curvature Ratio | | | | | | |
| Max von Mises | 1.2794 | 1.3342 | 1.3436 | 1.3251 | 1.3746 | 1.3108 |
| Stress (MPa) | | | | | | |
| Max principal | 1.1812 | 1.2111 | 1.1721 | 1.2232 | 1.2021 | 1.2059 |
| stress (MPa) | | | | | | |

TABLE 7
Young's Modulus: 50 GPa

| | | | | | | |
|---|---|---|---|---|---|---|
| Radius of | 1:1.0 | 1:1.5 | 1:2.0 | 1:2.5 | 1:3.0 | 1:3.5 |
| Curvature Ratio | | | | | | |
| Max von Mises | 319.84 | 333.56 | 335.91 | 331.26 | 343.65 | 327.70 |
| Stress (MPa) | | | | | | |
| Max principal | 295.29 | 302.77 | 293.01 | 305.79 | 300.52 | 301.48 |
| stress (MPa) | | | | | | |

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A medical implant having a center axis, the medical implant comprising:
a first flexible waved strand, disposed around the center axis; and
a second flexible waved strand, disposed around the center axis, and being in spatial communication with the first flexible waved strand to form a plurality of first unit shapes and a plurality of second unit shapes;
wherein the first unit shapes and the second unit shapes are staggered around the center axis, and
wherein the first unit shapes are coupled to the second unit shapes to cause the first and second flexible waved strands to move substantially along the center axis, the first and second flexible waved strands together define a self-anchoring configuration in a radial direction perpendicular to the center axis so that a ratio of a von Mises stress to an axial displacement of the medical implant during an implant compression of the medical implant is greater than 0.1 and less than 30, the von Mises stress is expressed in megapascals, and the axial displacement is expressed in millimeter.

2. The medical implant according to claim 1, wherein a radius of curvature of an outer edge of any curvilinear arc of the first flexible waved strand is less than or equal to a radius of curvature of an outer edge of any curvilinear arc of the second flexible waved strand.

3. The medical implant according to claim 2, wherein the radius of curvature of the outer edge of the curvilinear arc of the first flexible waved strand is less than 15 mm and not less than 0.35 mm.

4. The medical implant according to claim 2, wherein the radius of curvature of the outer edge of the curvilinear arc of the second flexible waved strand is less than 15 mm and not less than 0.35 mm.

5. The medical implant according to claim 1, wherein the first unit shape and the second unit shape are mutually exclusive in shape, and the first unit shape comprises two peaks and one trough of the first flexible waved strand.

6. The medical implant according to claim 5, wherein the trough has an internal angle that is less than 87 degrees and not less than 3 degrees.

7. The medical implant according to claim 5, wherein the peak has an internal angle that is less than 87 degrees and not less than 4 degrees.

8. The medical implant according to claim 5, wherein the first unit shape is heart-shaped.

9. The medical implant according to claim 8, wherein the second unit shape is reverse heart-shaped.

10. The medical implant according to claim 9, wherein the first unit shape has a first length along the center axis, the second unit shape has a second length along the center axis, and the first length is greater than the second length.

11. The medical implant according to claim 5, wherein the second unit shape is diamond.

12. The medical implant according to claim 11, wherein the first unit shape has a first length along the center axis, the second unit shape has a second length along the center axis, and the first length is less than the second length.

13. The medical implant according to claim 1, wherein the first unit shape and the second unit shape have different lengths along the center axis.

14. The medical implant according to claim 1, wherein the first flexible waved strand or the second flexible waved strand is made of biodegradable polymer, ceramic, metal alloy or a combination thereof.

15. The medical implant according to claim 1, wherein the first flexible waved strand or the second flexible waved strand comprises a plurality of filaments.

16. The medical implant according to claim 15, wherein the filament is biodegradable monofiber or multifiber.

17. The medical implant according to claim 15, wherein the plurality of filaments are twisted into a bundle with a predetermined section.

18. The medical implant according to claim 15, wherein the plurality of filaments are twisted into a bundle with a hexagon section.

19. The medical implant according to claim 15, wherein the filament is made of a polymeric material.

20. The medical implant according to claim 1, wherein the first flexible waved strand and the second flexible waved strand overlap and are connected through a plurality of joints, the joints are located between the first unit shapes and the second unit shapes, and a trough of the first flexible waved strand is aligned with a trough of the second flexible waved strand in a direction parallel to the center axis.

21. The medical implant according to claim 1, wherein the first flexible waved strand and the second flexible waved strand are connected through a plurality of joints at every two or more troughs of the first flexible waved strand and corresponding peaks of the second flexible waved strand.

22. The medical implant according to claim 1, wherein the axial displacement is 25% of a diameter of the medical implant.
